(19) United States
(12) Patent Application Publication
(10) Pub. No.: US 2005/0123521 A1
(43) Pub. Date: Jun. 9, 2005

(54) Immortalization of human cells by the ectopic expression of human telomerase reverse transcriptase

(75) Inventors: Mark A. Zern, Sacramento, CA (US); Henning Wege, Hamburg (DE)

Correspondence Address: TOWNSEND AND TOWNSEND AND CREW, LLP, TWO EMBARCADERO CENTER, EIGHTH FLOOR, SAN FRANCISCO, CA, 94111-3834, US

(73) Assignee: Regents of the University of California, Oakland, CA (US)

(21) Appl. No.: 10/481,191

(22) PCT Filed: Jun. 21, 2002
(86) PCT No.: PCT/US02/19639

Related U.S. Application Data
(60) Provisional application No. 60/300,181, filed on Jun. 21, 2001.
(60) Provisional application No. 60/390,520, filed on Jun. 21, 2002.

(51) Int. Cl. A61K 48/00; C12N 5/08; C12N 15/867

(52) U.S. Cl. 424/93.21; 435/366; 435/456; 435/368

(57) ABSTRACT

Immortalized primary adult and fetal human cells are provided that express a functional telomerase catalytic subunit from a nucleic acid sequence encoding the hTERT gene. The telomerase-immortalized human cells can be maintained in culture for at least 35 population doublings without developing a transformed phenotype, and while continuing to express proteins associated with the primary differentiated cell type. The invention is also directed to immortalized, non-transformed cell lines derived from primary human cells that express a functional telomerase catalytic subunit and to their use for transplantation to patients with hepatic disease. The invention also is directed to an improved biologic extracorporeal liver support device wherein the improvement is the use of telomerase-immortalized hepatocytes incorporated onto the scaffolding of the cell colonization chamber and use of the improved device for treating symptoms of patients with hepatic disease.

### FEDERALLY SPONSORED RESEARCH

This research was funded in part by a grant number R01 DK 6952 and P30 DK 41296 from the National Institutes of Health. The US government may have rights in this invention.

### INTRODUCTION

1. Field of the Invention

The invention relates to primary adult or fetal human cells made immortal through expression of a functional telomerase catalytic subunit. The invention is exemplified by immortalization of human fetal pancreatic islet cells and fetal hepatocytes through transduction with a human gene encoding a human telomerase reverse transcriptase catalytic subunit.

2. Background

Extracorporeal bioartificial organ support devices and cellular transplantation offer the possibility of effective treatment for many inherited and acquired organ disorders. Such therapeutic modalities could alleviate the crucial shortage of donor organs for whole organ transplantation. However, limited in vitro primary human cell proliferation has mandated that eeither less than adequate cell lines or animal cells be used for the development of extracorporeal organ support and cellular transplantation systems. Furthermore, the lack of donor organs or tissues makes it difficult to obtain enough viable human primary cells for the further advancement of cell-based transplantation therapies.

Various culture systems have already been evaluated, for instance complex media supplemented with mitogens (Block et al. (1996) J Cell Biol 132 (6): 1133-49; Runge et al., (2000) Biochem Biophys Res Commun 269: 46-53), dimethyl sulfoxide supplementation (Cable and Isom (1997) Hepatology 26: 1444-57), collagen gel sandwich techniques (Kono et al., (1995) Exp Cell Res 221: 478-85), and co-culture systems with non-parenchymal cells (Rojkind et al., (1995) Am J Pathol 146 (6): 1508-20). Cancer-derived cell lines, (Aden et al., (1979) Nature 282: 615-6; Sussmann et al., (1992) Hepatology 16 (1): 60-65), provide an endless supply of cells but often lack important metabolic and synthetic properties due to genetic alterations and carry the risk of tumor seeding to the patient (Javitt (1990) FASEB J 4: 161-68, Nyberg et al., (1994) Ann Surg 220(1):59-67).

One of the fundamental problems of in vitro primary cell propagation is that proliferation induces cellular dedifferentiation (i.e. specific functions associated with the differentiated cell are lost; for example, with cultured hepatocytes, the functions of albumin synthesis, cytochrome P450 oxidation, and ureagenesis are lost (Block, 1996, supra)). Non-human animal primary cells have the disadvantage that xenogenic proteins and enzymes enter the human blood circulation as well as posing a danger of xenozoonosis—the potential for transmission of animal virues, such as porcine viruses, including porcine endogenous retrovirus or hepatitis E virus (Patience et al., (1997) Nat Med 3(3):282-86, Meng et al., (1997) Proc Natl Acad Sci USA 94:9860-65). In addition, a small number of patients manifest a spontaneous cytotoxicity to animal derived cells such as porcine cells caused by natural xenoreactive IgM antibodies (Takahashi, et al. (1993) ASAIO J 39: M242-46).

Another alternative is to immortalize human primary cells by genetic engineering. Currently, different immortalization techniques are being investigated, for instance the introduction of the Simian virus 40 large T-antigen (Kobayashi et al., (2000) Science 287: 1258-62, Nakamura et al., (1997) Transplantation 63 (11): 1541-47), transfection of antisense constructions against p53 and retinoblastoma protein (Werner et al., (2000) Biotechnol Bioeng 68 (1): 59-70), transgenic introduction of a truncated Met protein (Amicone et al., (1997) EMBO J. 16 (3): 495-503), and expression of a hepatitis C virus core protein (Ray et al., (2000) Virology 271: 197-204).

Human cellular transplantation has been tried with varying degrees of success by several investigators. Most studies have employed the technique as a bridge to whole organ transplantation (see, for example, Mito, et al. (1993) Transplant 2: 65-74. Strom, et al. (1997) Transplant Proc 29: 2103-2106.). In several cases, the cellular transplantation has been associated with symptomatic improvement prior to organ transplantation. However, it is very difficult to assess whether the cellular transplantation was responsible for the improvements that occurred or whether the patients' native organs recovered and provided the improved function. A major issue in the human studies, as in the animal experiments, is the limited ability of most normal adult cells to proliferate and the limitation this creates when only a limited cell mass can be transplanted into a patient.

In the circumstances of acute organ failure, an effective extracorporeal organ support system that provides the patient with a bridge to organ transplantation, would dramatically improve the chances of survival. A few bioartificial organ system s have been shown to be safe in clinical trials, but their efficacy has not been proven. It therefore is of interest do develop extracorporeal bioartificial liver support devices and cells for transplantation that retain differentiated cell function and long-term cell-division capacities and do not carry the dangers associated with transformation or xenogenic sources.

Relevant Literature

Totsugawa, et al. published abstract for a poster presentation (#43) at the 47th American Society for Artificial Internal Organs (ASAIO) annual conference (Jun. 7-9, 2001 in New York, N.Y.) reporting transduction of human hepatocytes with an adenoviral vector that expressed the hTERT gene. No details on the transduction are provided. Initial clinical trials using non-immortalized human hepatocytes to repopulate diseased livers and correct metabolic disease are discussed in Ng, et al. (2000) Clin Liver Dis, 4: 929-945. See also Gupta, et al. (1999) J Gene Med 1: 386-392.

Bodnar, et al. report that expression of the human telomerase reverse transcriptase (hTERT) extends the life span of human fibroblasts and retinal pigment epithelial cells beyond senescence without causing neoplastic transformation (Science (1998) 279: 349-352). Yang, et al. disclose that introduction of the gene for hTERT into human large vessel and microvascular endothelial cells enables the cells to bypass replicative senescence without affecting their differentiation or causing the cells to exhibit a transformed phenotype (J Biol Chem (1999) 274: 26141-26148). Dickson, et al. disclose that human keratinocytes that express hTERT bypass a cell cycle checkpoint yet retain normal growth and differentiation characteristics (Mol Cell Biol 20: 1436-1447). Jiang, et al. report expression of the human telomerase catalytic component in human skin fibroblasts and retinal pigment epithelial cells, and that the cells retain normal growth control (Nature Genet (1999) 21: 111).

### SUMMARY OF THE INVENTION

The subject invention is directed to cells that have been immortalized by transduction with nucleic acid encoding a functional telomerase catalytic subunit together with methods of making and using the immortalized cells. The method of making the immortalized cells includes the steps of transducing isolated cells with a nucleic acid encoding a functional telomerase catalytic subunit and then growing the transduced cells in culture so that a functional telomerase catalytic subunit is expressed. A biologic extracorporeal cell support apparatus or bioartificial organ also is provided which contains the telomerase-immortalized cells. Also provided are methods of using the device to provide extracorporeal organ function to an individual in need thereof, the method including the step of perfusing whole blood, plasma or ultrafiltrate from the individual through the cell colonization chamber. Methods of treating the symptoms of an organ related disease in an individual include use of the bioartificial organ or directly transplanting the immortalized cells into the individual. The invention finds use in organ transplantation and in bioartificial organ assist devices as well as in differentiation and drug metabolism studies.

### DESCRIPTION OF THE DRAWINGS

FIG. 1. An illustration of the retroviral transfer system.

FIG. 2. Telomere length as determined by Southern blot analysis. Genomic DNA was digested with HinfI and RsaI, electrophoresed, blotted, and hybridized with ³²P-(TTAGGG)₃ as a telomeric probe. (A) Shown are representative TRF smears of control FH and FH-hTERT at different PD. The arrow indicates gel loading and the ruler DNA size markers in kb. (B) Mean telomere length was estimated based on weighted densitometric calculations, assuming a subtelomeric portion of 2.5 kb. Each data point represents the mean of 2 or more observations. Linear trendlines were added to visualize telomere length development. The first data point of control FH and FH-hTERT at 14 PD is 7.22±0.27 kb (mean±SEM; n=3).

FIG. 3. Representative proliferation curves of control FH and FH-hTERT pools generated from cell counts and plotted as PD vs. days post transduction. Each point represents the mean of 2 independent cell counts. The immortality threshold was set at twice the number of PD of replicative senescence.

FIG. 4. Senescence visualized in subconfluent, non-proliferating control FH cultures (30-35 PD) by blue senescence-associated β-galactosidase stain for 12-16 hours. No blue cells were observed in stained FH-hTERT cultures (data not shown). Note the large cytoplasma of senescent cells. (Original magnification 40×.).

FIG. 5. DNA-synthesis of control FH and FH-hTERT at differtent PD. Cells were labeled with 10 μmol/L BrdU for 2 hours and analyzed by flow cytometry. The bars show the mean percentage of cells with DNA synthesis for the indicated number of observations (above the bars)±SEM at different PD. (*FH-Control 30 PD vs. FH-hTERT 30 PD, P<0.001.).

FIG. 6. Hepatocellular gene expression of passaged FH (passage 1) and FH-hTERT at 50 PD as investigated by RT-PCR and standard agarose gel electrophoresis. Shown are the specific bands of the indicated targets (abbreviations as in Table 1) for representative reactions. RT-PCR reactions were repeated 2-4 times for verifying results.

FIG. 7. Glucose-6-phosphatase activity and glycogen storage of primary FH and FH-hTERT at 50 PD in confluent cultures. Phase contrast micrographs were taken from primary FH (A) and FH-hTERT (B). Glucose-6-phosphatase activity was visualized in primary FH(C) and FH-hTERT (D) by incubating with glucose-6-phosphate. For glycogen staining, ethanol-fixed primary FH (E) and FH-hTERT (F) were incubated with 1% aqueous periodic acid and Schiff's reagent each followed by washes in 0.5% sodium bisulphite water. (Original magnification 40×.).

FIG. 8. Western blot analysis of c-Myc in passaged FH and FH-hTERT at different PD. Employing Western blot analysis, c-Myc content was evaluated using passaged FH (Pass. FH) as calibrator and HeLa extract as a positive control. Shown are representative bands and their size in kilo Daltons (kDa) together with the densitometric values normalized to actin and relative to Pass. FH. The experiment was repeated twice to confirm the results.

FIG. 9. The panels show localization of transplanted cells by in situ hybridization using a human centromere probe labeled with digoxigenin. The detection system utilized the Fast Red substrate following immunostaining with an alkaline-phosphatase conjugated anti-digoxigenin antibody. (A) Positive control human liver showing visualization of nuclei. (B) Mouse liver showing absence of any hybridization signal (negative control, eosin counterstain to visualize cytoplasm. Nuclei appear as empty holes). (C and D) Liver from FH-hTERT transplanted mouse 3 days after cell. Transplanted cells contain red centromeric hybridization signals (arrows), with insets showing enlarged views of some of these transplanted cells. The transplanted cells were localized in portal areas (Pa) as expected. C and D were lightly counterstained with toluidine blue to stain nuclei.

FIG. 10. The data shown above are from NOD-SCID mice that were transplanted intraperitoneally with FH-hTERT cells after attaching cells to collagen coated microcarrier beads (mc). The microcarriers were recovered from animals after 4 weeks. Frozen sections were prepared and analyzed. The panel on the left shows cells with hepatocyte-like morphology with hematoxylin and eosin staining. The middle panel shows presence of glucose-6-phosphatase activity in transplanted cells. The panel on the right shows glycogen staining in transplanted cells.

FIG. 11. Bar graph depiction of the telomerase activity of nestin-positive islet-derived progenitor (NIP) cultures measured using a TeloTAGGG Telomerase PCR ELISA. Shown are averages of the indicated numbers of measurements±SEM for NIP and NIP hTERT during and after selection (NIP hTERT sel).

### DESCRIPTION OF THE SPECIFIC EMBODIMENTS

Immortalized mammalian cells, particularly hepatocytes or pancreatic cells, are obtained by the reconstitution of telomerase in the cells. Isolated cells, generally fetal or adult cells from a tissue or organ of interest, maintained in primary culture, are provided with nucleic acid encoding a telomerase catalytic subunit of telomerase reverse transcriptase (TERT). Transduction can be performed using for example a retroviral vector system or other viral vector such as SV40. By “primary” is intended that the cells have not been passaged in culture before being transduced with the hTERT gene, and includes both freshly isolated and cryopreserved cells. The cells generally are human as is the TERT gene.

The advantages of telomerase-immortalized cells include that they can provide all of the functions of a differentiated cell, without the undesirable risks associated with oncogene-immortalized cells or xenogenic cells. They do not develop a transformed or de-differentiated phenotype, even after extended population doublings. Using telomerase-immortalized cells, such as hepatocytes or pancreatic cells, for direct transplantation procedures has the benefit of greatly reduced cost, of providing endogenous organ function to a much greater number of individuals and of alleviating the overwhelming demand for whole organs (for example, a number of patients could be treated with one donated organ) without exposing the recipient to the morbidity and mortality associated with a full organ transplantation. An additional advantage of a cell-based replacement approach rather than organ transplantation is that the need for immunosuppresant therapy is substantially less and may be completely unnecessary over time because the immune response to a transplanted organ is primarily directed against epithelium and endothelial cells in the transplanted organ.

The cells for immortalization can be any derived from any tissue or organ and that would find use in extracorporeal or direct transplantation procedures, and include for example, liver cells, pancreatic β-cells, dopaminergic neurons, adrenocortical cells, lung, kidney cells, pituitary gland cells. The cells are isolated using standard procedures and either cryopreserved or maintained in primary culture until use. Of particular interest are liver and pancreatic cells. Primary adult hepatocytes are harvested from human cadavers. Conveniently they can be obtained from the Liver Tissue Procurement and Distribution System, Minneapolis, Minn., and maintained in primary culture following standard protocols (Runge et al., 2000) until immortalization. Primary fetal hepatocytes are harvested from aborted human fetuses, preferably from the second trimester of gestation. Procedures for isolating and culturing epithelial cells from human fetal livers are described in Zahler, et al. (2000) J Gene Med 2: 186-193. As a source of pancreatic cells, pancreatic P-cells, nestin-positive islet-derived progenitor cells from adult or fetal sources, or biopsies of diabetic patients can be used.

The human telomerase catalytic subunit has been cloned (see Nakamura, et al. (1997) Science 277: 955; Mayerson, et al. (1997) Cell 90: 78; and Kilian, et al. (1997) Hum Mol Genet 6: 2011; U.S. Pat. No. 6,166,178). Sources of the coding sequence for the human telomerase subunit include any cells that demonstrate telomerase activity such as immortal cell lines, tumor tissues, germ cells, proliferating stem or progenitor cells, and activated lymphocytes. The nucleic acid can be obtained using methods known in the art.

The manner in which the hTERT coding region is introduced into the cells of interest is not critical, as long as a functional telomerase catalytic subunit is expressed. Expression can be extrachromosomal or following integration into the celluluar genome. Any of a variety of techniques can be used to introduce the hTERT gene into the desired cells, including electroporation, liposomes, or viral vectors. See Molecular Cloning, 3^{rd} Edition, 2001, by Sambrook and Russell. Generally the hTERT coding sequence is introduced using a viral vector, for example SV40, adenovirus, Herpes simplex virus, adeno-associated virus, and the like. See Blomer et al., Human Molecular Genetics 5 Spec No: 1397-404, 1996; Zern et al., Gene Ther. 6: 114-120, 1999; and Robbins et al., Trends in Biotechnology 16: 35-40, 1998. Each of these systems has a different host range and can be used to infect cells that are refractory to retrovirus expression (see below), i.e., non-dividing cells.

A preferred means for incorporating the hTERT coding region into the cells of interest is to use a recombinant retrovirus that provides for integratration of the hTERT efficiently and stably into the genome of the target cell. Since the intended use of the immortalized cells is in human therapy, it is important that the retrovirus used is replication-defective and not contaminated with wild-type viruses (Temin, (1986) in Gene Transfer, R. Kucherlapati, eds. Plenum Press, New York, p. 149 and Temin, (1990) Hum. Gene Ther. 1: 111). The recombinant retroviruses which are used are derived from viruses with a natural host-specificity that includes primates, or from viruses that can be pseudotyped with a host-specificity that includes primates. Such viruses include, murine leukemia viruses (MuLV; Weiss et al., (1984) RNA Tumor Viruses, New York) with a so-called amphotropic or xenotropic host-range, gibbon ape leukemia viruses (GaLV; Lieber et al., Proc. Natl. Acad. Sci. USA 72 (1975) 2315-2319), and primate lentiviruses. For the production of recombinant retroviruses, two elements are required: the so-called retroviral vector, which, in addition to the gene (or genes) to be introduced, contains all DNA elements of a retrovirus that are necessary for packaging the viral genome and the integration into the host genome; and the so-called packaging cell line which produces the viral proteins that are necessary for building up an infectious recombinant retrovirus (Miller, (1990), Hum. Gene Ther. 1: 5).

The packaging cell lines used should be those constructed so that the risk of recombination events whereby a replication-competent virus is generated, are minimized. This generally is effected by physically separating into two parts the parts of the virus genome that code for viral proteins and introducing them into the cell line separately (Danos and Mulligan, (1988) Proc. Natl. Acad. Sci. USA 85: 6460; Markowitz et al., (1988) J. Virol. 62: 1120; and Markowitz et al., (1988) Virology 167: 400). As the presence of both constructs is essential to the functioning of the packaging cell line and chromosomal instability occurs regularly, it is important for use of such cells in procedures related to human therapy that, by means of a selection medium, selection for the presence of the constructs can be provided for. Therefore, these constructs generally are introduced by means of a cotransfection whereby both viral constructs are transfected together with a dominant selection marker.

The retroviral vectors generally include as operatively linked components, retroviral long terminal repeats, packaging sequences and cloning site(s) for insertion of heterologous sequences. Other operatively linked components may include a nonretroviral promoter/enhancer and a selectable marker gene. Examples of retrovirus expression vectors which can be used include DC-T5T (Sullenger et al. 1990. Mol. Cell Biol. 10: 6512-65230), kat (Blood. 1994 83: 43-50), BOSC (Proc. Natl. Acad. Sci. (USA) (1993) 90: 8392-8396), pBabe (Proc. Natl. Acad. Sci. (USA) (1995) 92: 9146-9150) and RetroXpreSS™ (Clontech, Palo Alto, Calif.). An expression vector is available that includes the hTERT gene, for example pBabe-puro-hTERT (Morgenstern and Land 1990). In some instances, it may be desirable to increase expression of the hTERT gene by utilizing other promoters and/or enhancers in place of the promoter and/or enhancers provided in the expression vector. These promoters in combination with enhancers can be constitutive or regulatable. Any promoter/enhancer system functional in the target cell can be used. (See for example, Molecular Virology pp. 176-177; Hofmann, et al. 1996. Proc. Natl. Acad. Sci. (USA) 93: 5185-5190; Coffin and Varmus, 1996. Retroviruses. Cold Spring Harbor Press, NY; Ausubel et al. 1994. Current Protocols in Molecular Biology. Greene Publishing Associates, Inc. & Wiley and Sons, Inc.). Examples include: CMV immediate-early promoter, SV40, thymidine kinase promoter, metalothionine promoter, and tetracycline operator (Hofman et al., (1996) Proc. Natl. Acad. Sci (USA) 93: 5185-5190).

To package the recombinant retrovirus vectors containing the nucleic acid-to-be-expressed, cells lines are used that provide in trans the gene functions deleted from the recombinant retrovirus vector such that the vector is replicated and packaged into virus particles. The genes expressed in trans encode viral structural proteins and enzymes for packaging the vector and carrying out essential functions required for the vector's expression following infection of the target host cell. Packaging cell lines and retrovirus vector combinations that minimize homologous recombination between the vector and the genes expressed in trans are preferred to avoid the generation of replication competent retrovirus. Packaging systems that provide essential gene functions in trans from co-transfected expression vectors can be used and packaging systems that produce replication competent retroviruses. Following packaging, the recombinant retrovirus is used to infect target cells of interest. The envelope proteins expressed should permit infection of the target cell by the recombinant retrovirus particle. Retrovirus packaging cell lines which can be used include BOSC23 (Proc. Natl. Acad. Sci. (USA) 90: 8392-8396), PT67 (Miller and Miller. 1994. J. Virol. 68: 8270-8276, Miller. 1996. Proc. Natl. Acad. Sci. (USA) 93: 11407-11413), PA317. (Mol. Cell Biol. 6: 2895 (1986)), PG13, 293 cells transfected with pIK6.1 packaging plasmids (U.S. Pat. No. 5,686,279), GP+envAM12 (Virology 167: 400 (1988), PE502 cells (BioTechniques 7: 980-990 (1989)), GP+86 (Markowitz, et al. 1988. J. Virol. 62: 1120-1124), Ψ-Cre (Danos and Mulligan. 1988. Proc. Natl. Acad. Sci. (USA) 85: 6460-6464). The preferred titer of recombinant retrovirus particles is about 10⁵-10⁷ infectious particles per milliliter. If these titers cannot be achieved the virus also can be concentrated before use.

For transfection, the hepatocytes or other cells to be transfected are suspended in a suitable culture medium containing recombinant retrovirus vector particles. Many different suitable culture media are commercially available. They include DMEM, IMDM, and α-MEM, with 5-30% serum and often further supplemented with, e.g., BSA, one or more antibiotics and optionally growth factors suitable for stimulating cell division. Recombinant retrovirus vector particles are harvested into this medium by incubating the virus-producing cells in this medium. To enhance gene transfer, compounds such as polybrene, protamine sulphate, or protamine HCl generally are added. Usually, the cultures are maintained for 2-4 days and the recombinant retrovirus vector containing medium is refreshed daily. Optionally, the cells to be transfected are precultured in medium with growth factors but without recombinant retrovirus vector particles for up to 2 days, before adding the recombinant retrovirus vector containing medium. For successful gene transfer it is essential that the target cells undergo replication in culture (without differentiation).

Successfully transduced cells are selected by culturing cells in medium containing a selection drug (puromycin, hygromycin, G418) that allows permissive growth only by cells that express an appropriate selection marker gene, and are analyzed for mRNA levels of the telomerase catalytic unit, using RT-PCR, particularly real-time RT-PCR oftentimes used in evaluating telomerase activity, or by using commercially available kits (Roche Molecular Biochemicals) or other techniques known in the art.

Expression of the telomerase catalytic unit is confirmed using flow cytometry (Ali, et al. (2000) Leukemia 14: 2176-2181). Telomerase activity of transfected cells is determined using any of the myriad variations of telomeric repeat amplification protocol (TRAP) assays in the literature and known to those in the art. Either a non-amplified or a PCR-based assay can be applied (Kim and Wu 1997). TRAP assays that utilize radiochemical—(i.e. ³²P) or enzyme-(ELISA) based detection can be applied. Telomere length comparisions between transduced and non-transduced cells are carried out by isolating genomic DNA and then digesting with a restriction enzyme that does not cut within the telomeres (for example, HinfI and RsaI). The undigested telomeres are then labeled (with a radiochemical, a fluorescent compound, or an enzyme) and resolved in a gel. In applications where it is desired, once it is established that the hTERT coding sequence is incorporated into the genomic DNA, it is preferred to maintain the cells in the absence of selective drug. The selective drug is removed before the cells are used therapeutically.

To confirm that the transduced cells have been immortalized, the phenotype of hTERT-transduced cells is evaluated and compared to non-transduced cells, and transformed immortal cells. Transduced cells are less susceptible to induction of apoptosis and do not develop staining characteristics associated with senescence, retaining normal chromosome patterns. Telomerase immortalized cells do not acquire morphologic or phenotypic changes generally associated with cancer cells (they maintain checkpoint control of growth, do not grow in soft agar, and retain normal pRb phosphorylation patterns in response to confluence-induced growth arrest) (Yang et al. (1999) J. Biol Chem 274: 26141; Bodnar et al. (1998) Science 279: 349), yet their growth curves are similar to those of transformed cell lines.

Proliferative capacity of the transduced cells is compared to untransduced control cells. For example, the cells are grown in monolayers until cell cycle arrest or immortality can be confirmed (2-fold increase in doubling potential). The number of population doublings (PD) is estimated by the count/split-method (Vaziri and Benchimol S (1998) Curr Biol 8: 279-282). Growth curves are generated for the cultured cells and time to confluency is determined. β-galactosidase can serve as a biomarker to visualize senescence in hepatocyte cultures (Dimri et al (1995) Proc Natl Acad Sci USA 92: 9363-9367). ³H-thymidine incorporation (18) and a BrdU incorporation-based flow cytometry assay (BrdU Flow Kit, PharMingen) are used to detect DNA synthesis and to characterize the cell cycle distribution of the cultured cells.

Hepatospecific gene expression, including albumin, α-fetoprotein, α1-antitrypsin, transferrin, and different cytochrome P450 subtypes, of the immortalized cells is compared to freshly isolated human hepatocytes by real-time quantitative RT-PCR using Taqman chemistry (PE Applied Biosystems). Also, protein synthesis is measured by Western immunoblotting. Inducibility of cytochrome P450 2E1 subtype is evaluated by treating the cells with ethanol prior to expression analysis. Ureagenesis is evaluated for example in a serum-free incubation system by measuring urea production and ammonium removal from the culture medium. Pancreatic cell expression of markers, including GLUT2, insulin, glucagon, and the homeodomain transcription factor IDX-1, of the immortalized cells is compared to freshly isolated human pancreatic cells by real-time quantitative RT-PCR using Taqman chemistry (PE Applied Biosystems).

Growth requirements, karyotypic stability, and cell-cycle checkpoints are analyzed to detect any possible oncogenic potential of the immortalized cells using soft-agar colony assays and culture in a low-serum medium. The presence of chromosomal abnormalities is a fundamental feature of tumor progression. Karyotype analysis is conducted by G-banding in a cytogenetic laboratory. Western analysis for the hypo- and hyperphosphorylated forms of retinoblastoma protein are performed after cell growth in low-serum medium (Jiang X R, Jimenez G, Chang E, Frolkis M, Kusler B, Sage M, Beeche M, Bodnar A G, Wahl G M, Tlsty T D, Chiu C P (1999) Telomerase expression in human somatic cells does not induce changes associated with a transformed phenotype. Nat Genet 21: 111-114). Finally, DNA damage is induced by UV-B irradiation (100 J/m²) and p53 is quantitated by Western immunoblotting.

Telomerase-immortalized human cells can be used to treat symptoms associated with the failure of differentiated organs and tissues that are amenable to organ or tissue transplantation. The immortalized cells can be used for transplantation into a patient in need thereof or, as appropriate, can be used as part of an extracorporeal organ support and direct cell transplantation treatments. Conditions for which the telomerase-immortalized cells can be used include liver failure, pancreatic failure (for example, loss of capability for responsiveness to increasing blood glucose concentrations and/or insulin production), kidney failure, Parkinson's disease, adrenal insufficiency, pituitary insufficiency, and the failure of other endocrine organs. Of particular interest is the treatment of symptoms of acute or chronic liver failure, due to for example fulminant hepatic failure (FHF), decompensated cirrhosis, drug overdose or other corporeal poisoning, or hepatic failure due to disease, such as hepatitis or cancer. Also of particular interest is the treatment of symptoms of either of the two major forms of diabetes mellitus: insulin-dependent (Type 1) diabetes mellitus (IDDM) and non-insulin-dependent (Type 2) diabetes mellitus (NIDDM) which comprises roughly 90% of cases. In both Type 1 and Type 2 cases, there is a loss of insulin secretion, either through destruction of the p-cells in the pancreas or defective secretion or production of insulin, which can be remedied either through transplantation of immortalized pancreatic cells and/or by use of a bioartificial pancreas.

In the construction of a bioartificial organ, telomerase immortalized cells are inoculated into a chamber containing scaffolding structures designed to harbor high densities of viable immobilized cells. Cell scaffolding structures included in such devices are known to those of skill in the art, and include hollow fibers, dialysis capillaries, collagen sheets, spherical particles, encapsulated particles and foams. Examples of such chambers are ELAD™ by VitaGen of La Jolla, Calif.; Ellis, et al. (1996), HepatAssist® by Circe Biomedical of Lexington, Mass. (Watanabe, et al. (1997) Ann Surg 225: 484-494), those manufactured by Excort, Algenix, and HemoTherapies. In use, the modules of the bioartificial organ colonized with the immortalized cells are perfused with whole blood, plasma, or ultrafiltrate from the individual in need of treatment. In the case of a bioartificial liver, exogenous (i.e. drugs, poisons) and endogenous (ammonia, glutamine, bile acids, lactate, etc) toxins are removed and nutrients (i.e. glucose) are replenished before the perfusion is returned to the patient.

Telomerase immortalized-transplantable human cells also can be used in cellular transplantation procedures. In carrying out cellular transplantation, a sufficient number of immortalized cells to enable functional repopulation of a compromised organ or tissue are injected directly into the individual requiring treatment. In the application of telomerase-immortalized hepatocytes to direct hepatocyte transplantation, immortalized hepatocytes, generally in the amount of about 10% of a normal liver mass are injected intravenously (i.v.), intraperitoneally (i.p.), intrasplenically (i.s.), or directly intrahepatically (i.h.) into the patient in need thereof. Where the number of cells in a normal adult liver are estimated to be about 2.5×10¹¹ to 3.5×10¹¹ total cells, up to about 2.5×10¹⁰ to 3.5×10¹⁰ telomerase immortalized hepatocytes are injected in a cellular transplantation procedure. Depending on the size of the liver, the individual and the condition being treated, a lesser or greater number of cells are injected. The cells are administered in at least one treatment, but can be administered over several treatments. A maximum number of cells (i.e. about 10%) or a fraction of the maximum number of cells (up to 10%) are administered in each of one or more treatments. Generally, one treatment is sufficient for the immortalized hepatocytes to proliferate and appropriately associate themselves with the endogenous liver tissue, such that normal liver function is regenerated, even if the endogenous liver tissue does not itself regenerate. Additional treatments are administered if necessary.

Following treatment, the patient is evaluated to determine whether symptoms have been alleviated. Both the biological efficacy of the treatment modality as well as the clinical efficacy are evaluated, if possible. The clinical efficacy, i.e. whether treatment of the underlying effect is effective in changing the course of disease, can be more difficult to measure. While the evaluation of the biological efficacy goes a long way as a surrogate endpoint for the clinical efficacy, it is not definitive. Thus, measuring a clinical endpoint which can give an indication of the presence of functioning immortalized cells after, for example, a six-month period of time, can give an indication of the clinical efficacy of the treatment.

For a patient treated extracorporeally for liver disease, the evaluation can include any combination of the following depending upon the patient's presenting symptoms: plasma ammonia and lactate levels, serum transaminase levels, encephalopathy, and intracranial pressure (ICP), and serum glucose, systolic blood pressure and cranial perfusion pressure levels (Hepatology (1993) 17: 258-265) as compared to prior to receiving treatment. Following transplantation, liver function tests are performed to evaluate the sufficiency of the treatment in restoring substantially normal liver function. The liver function tests performed will depend upon the patient's original diagnosis, but generally will include one of more for the following tests: betransaminase (alanine aminotransferase) levels, creatinine clearance and bilirubin values (see Harrison's Principles of Internal Medicine, 15^{th} Edition, 2001). Furthermore, the ability to carry out normal hepatic metabolic functions such as those provided by the cytochrome P450 system, amino acid and gluconeogenesis, albumin synthesis, detoxification (i.e. glucuronidation) pathways, and plasma ammonium levels can be evaluated as appropriate. Standard liver function tests are well known to those in the art. Post-cellular transplant plasma levels of toxic metabolites that accumulate when liver functioning is compromised, such as for example, ammonium, aromatic amino acids, glutamine, endogenous benzodiazepine-like substances, neurosteroids, octopamine, GABA, serotonin, 2-oxindole and other tryptophan metabolites, bile acid, ketones, lactate, manganese, mercaptans and phenyls also can be evaluated.

For a patient treated for diabetes the treatment can be evaluated as follows. For example, since the disease manifests itself by increased blood sugar, the biological efficacy of the treatment therefore can be evaluated by observation of return of the evaluated blood glucose towards normal. Where it is desired to treat a particular diabetic complication, both the biological efficacy of the treatment modality as well as the clinical efficacy are evaluated, if possible. For example, in the treatment of diabetic cardiomyopathy, which manifests itself by subclinical abnormalities of left-ventricular diastolic function, such as impaired left-ventricular relaxation on digitized M-mode or Doppler echocardiography, the biological efficacy of the treatment can be evaluated, for example, by observation of return of the impaired left-ventricular relaxation to normal or cessation of progression. Similarly, in the treatment of another diabetic complication, diabetic retinopathy, therapy can be evaluated by before and after treatment observations of changes in the disease manifestations. Diabetic retinopathy is divided into three levels—(1) no diabetic retinopathy, (2) nonproliferative diabetic retinopathy, and (3) proliferative diabetic retinopathy. In addition, macular edema can be present at any level of retinopathy and is also graded. No retinopathy refers to a clinically normal fundus. Nonproliferative retinopathy is subdivided into four groups: mild, moderate, severe and very severe. Each level is precisely defined by the presence and degree of specific clinical features—hemorrhages with or without micoaneurysms, venous bleeding, and intraretinal microvascular abnormalities (see Early Treatment Diabetic Retinopathy Study Report Number 10: Grading diabetic retinopathy from stereoscopic color fundus photographs—an extension of the modified Airlie House classification. Opthalmology 98: 786-806, 1991). Proliferative retinopathy indicates the presence of any retinal neovascularization. The extent and progression or regression of diabetic retinopathy are evaluated and monitored by fundus photographs, direct or indirect ophthalmoscopy, and fluorescein angiography of the retina. The presence of retinal thickening within the macula constitutes macular edema. This is assessed by slit-lamp microscopy. In terms of clinical features of diabetic retinopathy, even extensive proliferative changes may cause no visual symptoms until vitreous hemorrhage or retinal detachment occurs. Retinal detachment not observable due to vitreous hemorrhage can be detected by B-scan ultrasonography. Macular edema causes a non-correctable decline in visual acuity. Absence of progression or regression of these changes after a period in which they generally would be expected to develop in a patient with a particular level of diabetic retinopathy can give an indication of the clinical efficacy of the treatment regimen. The manifestations and clinical features of other diabetic complications are described in Brownlee and King, Endocrinology and Metabolism Clinics of North America, 25: 1-483, 1996.

The following examples are offered by way of illustration of the present invention, not limitation.

### EXAMPLES

### Example 1

### Isolation of Human Adult and Fetal Hepatocytes

Isolated human hepatocytes are obtained from an NIH-supported Liver Tissue Procurement and Distribution System and maintained in primary culture following standard protocols (Runge et al (2000) Biochem Biophys Res Commun 269: 46-53). Fetal tissues are received from programs approved by the IRBs at Albert Einstein College of Medicine and Montefiore Medical Center in the Bronx, including from the Fetal Tissue Repository established by the College. Fetal livers were digested in 0.05% collagenase (Worthington Biochemical Corporation, Lakewood, N.J.) for 30-40 minutes at 37° C. Dissociated cells were passed through 80-μm dacron mesh, washed, and pelleted at 500×g for 4 minutes at 4° C. The cell pellet was resuspended in Dulbecco's modified Eagle's medium (Invitrogen, Carlsbad, Calif.), containing 5 μg/mL insulin (Sigma, St. Louis, Mo.), 2.4 μg/mL hydrocortisone (Sigma), 10% inactivated fetal bovine serum (Invitrogen), and standard antibiotics. Primary cultures were established in tissue culture dishes at 4×10³ cells per cm² in a humidified 5% CO₂ atmosphere. Cultures were passaged at a ratio of 1:4 thereafter. Fetal liver cells are either freshly isolated or cryopreserved for shipment.

### Example 2

### Cell Transduction and Selection

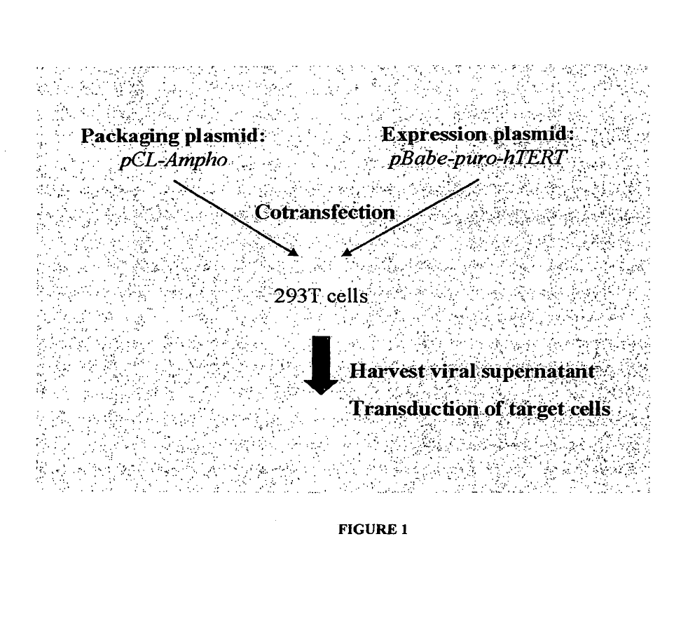

To produce retroviral vectors, 293T cells were cotransfected with the amphotropic packaging plasmid pCL-Ampho (Naviaux et al. J Virol 1996; 70: 5701-5705; Imgenex, San Diego, Calif.) and the expression plasmid pBabe-puro-hTERT or the control plasmid pBabe-puro (Counter et al. Proc Natl Acad Sci USA 1998; 95: 14723-14728) in 100-mm tissue culture dishes (pBabe-puro plasmids and the 293T cell line were kindly provided by Dr. R. A. Weinberg, Whitehead Institute for Biomedical Research, Cambridge, Mass.). The expression plasmid pBabe-puro-hTERT contains hTERT and puromycin acetyltransferase as a selectable marker under the control of a Moloney murine leukemia virus-derived promoter (see FIG. 1). FuGene 6 (Roche Molecular Biochemicals, Indianapolis, Ind.) was used for cotransfection according to the manufacturer. After 48 and 72 hours, viral supernatants were purified by passing through a 0.45-μm filter and used for immediate transduction in the presence of 8 μg/mL hexadimethrine bromide (Sigma). The titer of the supernatant was 2×10⁴ transducing units/mL, as determined by bioassay. On four consecutive days, passaged FH were transduced for 3 hours daily, corresponding to a total multiplicity of infection of 0.5. Following transduction and recovery for 24 hours, cells were selected for 7 days with 0.75 μmol/L puromycin dihydrochloride (Sigma).

### Example 3

### Telomerase Activity and Telomere Length Following hTERT-Transduction

hTERT expression was evaluated by real-time quantitative reverse transcription polymerase chain reaction (RT-PCR). Total RNA (150 ng), extracted with TriReagent-LS (Molecular Research Center, Cincinnati, Ohio), was used for the LightCycler TeloTAGGG hTERT Quantification Kit (Roche Molecular Biochemicals). As suggested in the kit manual, hTERT expression was normalized to the housekeeping gene porphobilinogen deaminase. The TeloTAGGG Telomerase PCR ELISA^{PLUS} (Roche Molecular Biochemicals) was used to measure telomerase activity in cellular extracts relative to a control template of 0.1 amole telomeric repeats. The telomerase activity assay employs the PCR-based telomeric repeat amplification protocol (Kim et al. Science 1994; 266: 2011-2015) and utilizes an enzyme-linked immunosorbent assay for quantification. The results were expressed as total product generated per μg of cellular protein and a telomere-primer elongation time of 20 minutes (Kim and Wu Nucleic Acids Res 1997; 25: 2595-2597).

Mean telomere length was analyzed by Southern blotting. Genomic DNA was extracted with DNAzol (Invitrogen), digested with HinfI and RsaI, electrophoresed, blotted, and transferred to positively-charged Magnacharge membranes (Osmonics, Minnetonka, Minn.) by alkaline blotting as described (Nichols et al. Scand J Immunol 1999; 49: 302-306). Membranes were hybridized with ³²P-(TTAGGG)₃ as a telomeric probe using Hybrisol II (Intergen, Purchase, N.Y.) and washed following published protocols. Mean terminal restriction fragment (TRF) length was determined from scanned autoradiographs by integrating the signal intensity above background over the entire TRF distribution using ImageQuaNT software (Molecular Dynamics, Sunnyvale, Calif.). In brief, TRF smears were divided into 30 equally sized boxes and the signal intensity within each box OD_{b} was used together with the molecular weight at the mid-point of the box MW_{b} to compute mean TRF length as L=Σ(OD_{b}XMW_{b})/Σ(OD_{b}) (Vaziri et al. Am J Hum Genet 1993; 52: 661-667). A subtelomeric portion of 2.5 kb was assumed to convert mean TRF length to mean telomere length (Counter et al. EMBO J. 1992; 11: 1921-1929).

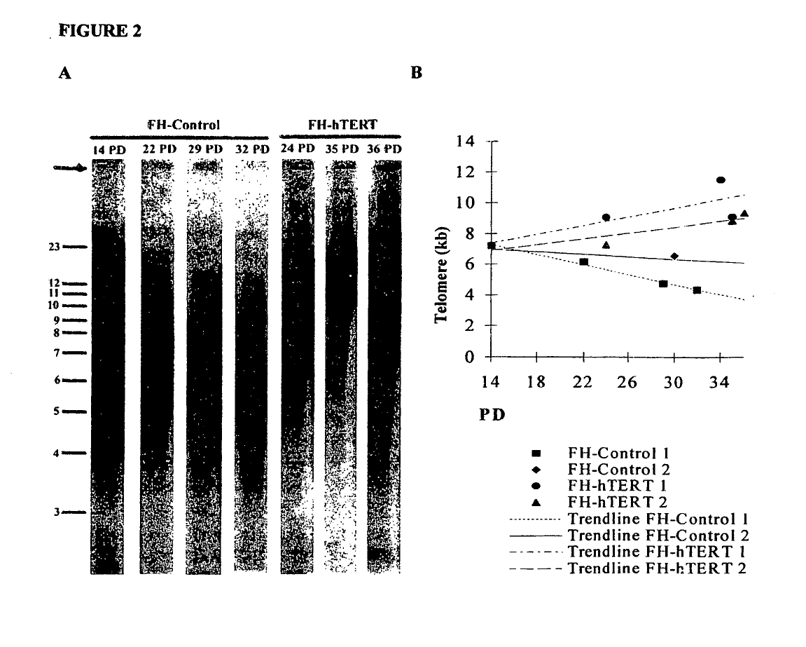

Isolated hepatocytes from a fetal human liver were cultured and transduced with hTERT or a control vector without hTERT at passage 4. Following puromycin selection, high levels of hTERT mRNA, 18.96±3.80 hTERT copies per copy of porphobilinogen deaminase (n=5), were detected in hTERT-transduced cells (FH-hTERT) using real-time quantitative RT-PCR. In comparison, no hTERT message was amplified in either untransduced FH (n=4) or cells transduced with the control vector (n=2). Furthermore, telomerase activity, as assessed by the telomeric repeat amplification protocol, increased significantly from 0.17±0.05 (n=4) to 65.75±4.49 total product generated·μg⁻¹·20 min⁻¹(n=5; P<0.001), levels that were similar to HepG2 and other telomerase-positive cell lines. The low telomerase activity in control FH was not consistently detected and presumably due to occasional telomerase positive cells. No increase in telomerase activity was detected in FH transduced with the control vector (n=2). Analyses of telomerase activity after 50 PD demonstrated that telomerase activity did not decrease in FH-hTERT despite continuous cell proliferation during long-term culture. Having established that hTERT-transduction successfully reconstituted telomerase activity in FH, we assessed telomere length by Southern blot analysis to investigate whether telomerase reconstitution resulted in telomere stabilization. As expected, FH-hTERT preserved elongated telomeres, while the mean telomere length in untransduced control cells shortened progressively to less than 6 kb after 25 PD. This is illustrated by representative TRF smears (FIG. 2A) and the linear trendlines of mean telomere length development (FIG. 2B). In comparison, the mean telomere length of senescent human fibroblasts in vitro was reported to be 6 kb (Allsopp et al. Proc Natl Acad Sci USA 1992; 89: 10114-10118; Campisi J. et al. Eur J Cancer 1997;33:703-709; Harley, Mutat Res 1991; 256: 271-282). According to our data, the telomere loss in control FH was 50 to 150 bp per PD (FIG. 2B), comparable to reported telomere erosion rates for human fibroblasts.

### Example 4

### Assessment of Proliferative Capacity

Cells were passaged when confluent and the number of population doublings (PD) was calculated as PD=log (N_{f}/Nᵢ)/log 2, where N_{f} is the final number of cells and Nᵢ the number of cells initially seeded (Vaziri and Benchimol. Curr Biol 1998; 8: 279-282). No correction was made for dead cells or cells that failed to reinitiate growth at subculture. To measure the percentage of cells in S-phase (DNA synthesis), 10⁶ cells were cultured overnight in 25-cm² tissue culture flasks and labeled for 2 hours with 10 μmol/L 5-bromo-2′-deoxyuridine (BrdU) on the following day. BrdU-incorporation was detected with the BrdU Flow Kit (BD PharMingen, San Diego, Calif.) as recommended in the kit manual using a FACScan flow cytometer and CellQuest software (BD Immunocytometry Systems, San Jose, Calif.). To visualize senescent cells, subconfluent cultures were fixed with 0.2% glutaraldehyde in 2% formaldehyde and stained in a humidified chamber at 37° C. for 12-16 hours. The staining solution contained 1 mg/mL 5-bromo-4-chloro-3-indolyl β-D-galactopyranoside (Sigma) in a staining buffer described previously.²⁶ Stained cell populations were evaluated by light microscopy and considered senescent if more than 90% of cells exhibited the characteristic blue senescence-associated β-galactosidase stain.

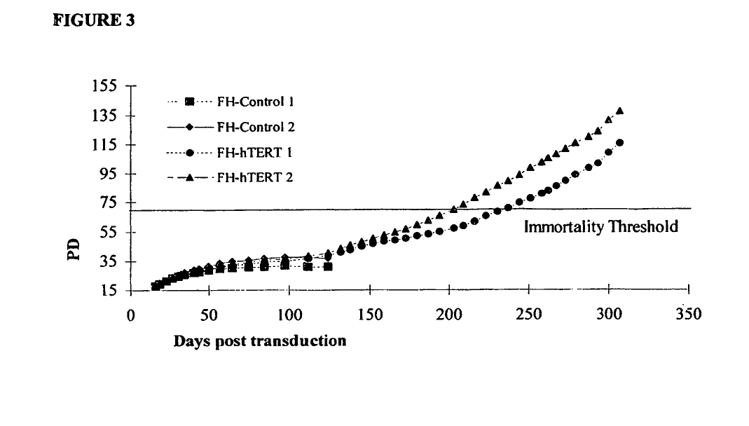

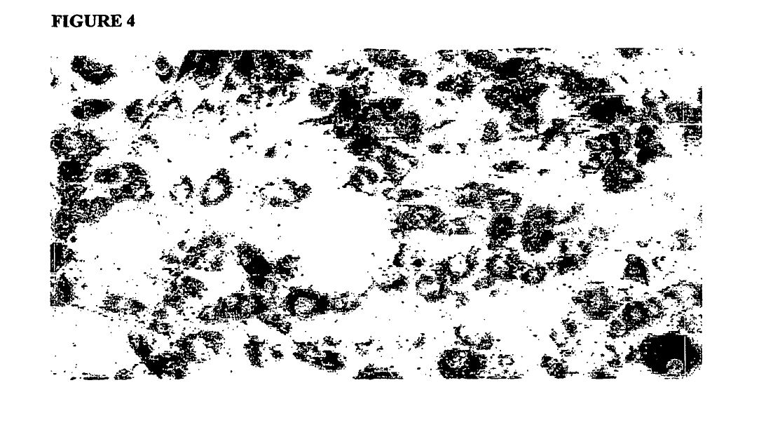

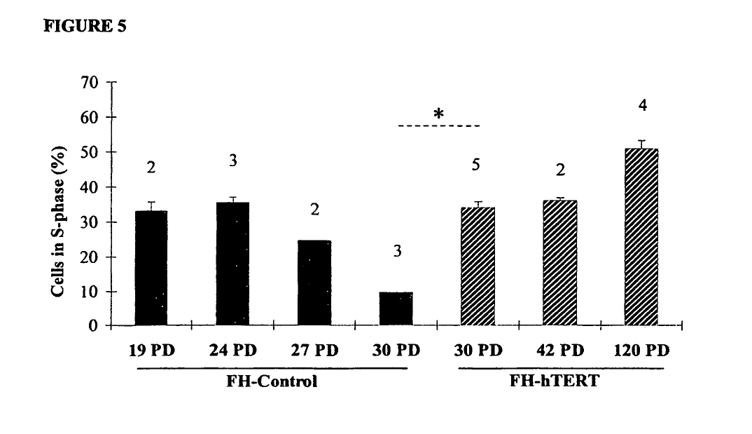

To examine the ability of FH-hTERT to bypass replicative senescence, we cultured control FH and FH-hTERT, and determined cell number and speed of proliferation with every subpassage. Altogether, 5 FH-hTERT and 6 control FH (4 untransduced and 2 control vector-transduced) cultures were observed and followed. After 30-35 PD, proliferation virtually ceased in control FH (FIG. 3) with entry into replicative senescence, as suggested by the appearance of cells with extremely large cytoplasma and a positive senescence-associated □-galactosidase stain (FIG. 4). FH-hTERT proliferation accelerated approaching senescence and continued beyond replicative senescence for more than 250 PD (to date). The speed of FH-hTERT proliferation was similar during the initial passages and advancing immortality. The immortality threshold, set at twice the number of PD of replicative senescence, was reached 200-250 days post transduction (FIG. 3). To further ascertain changes in the cell division potential, the percentage of cells in S-phase was measured. Approaching senescence (27 PD and 30 PD; FIG. 5), the percentage of cells in S-phase decreased in control FH. After 30 PD, which represents a time close to senescence or near-senescence with less than one PD per week (Bodnar et al. Science 1998; 279: 349-352), the percentage of cells in S-phase was significantly lower in control FH than FH-hTERT (9.62±0.03%; n=2 vs. 34.06±1.69%; n=5; P<0.001; FIG. 5).

All values in the examples are represented as means±SEM. The number of observations is stated in the text and figures. The unpaired Student's t test was used for statistical analysis and P<0.05 was considered significant.

### Example 5

### Evaluation of Hepatospecific Functions

For the evaluation of hepatic growth factors, growth factor receptors, and transcription factors, total RNA (1 μg), extracted with TRIzol (Invitrogen), was analyzed by one-step RT-PCR employing the GeneAmp Gold RNA PCR Kit (Applied Biosytems, Foster City, Calif.) with 0.2 mmol/L forward and reverse primers. Primer sequences and RT-PCR parameters are listed in Table 1. All samples, including no template controls, were subjected to 35 cycles and resolved by standard agarose gel electrophoresis. To detect albumin synthesis, ethanol-fixed cells were incubated with 1:100 diluted monoclonal anti-human serum albumin antibody clone HSA-11 (Sigma) or an equal immunoglobulin concentration of IgG2a isotype control (Sigma) for 4 hours at 4° C. Albumin-positive cells were detected after staining with 1:5,000 diluted anti-mouse immunoglobulin conjugated with fluorescein isothiocyanate (Sigma), 45 minutes at 4° C., using the flow cytometry equipment listed above. Glucose-6-phosphatase activity was visualized in unfixed cells by incubating with glucose-6-phosphate and post-fixation with ethanol essentially as described previously (Ott et al. J. Pathol 1999; 187: 365-373). For glycogen staining, cells were fixed in cold ethanol for 10 minutes at room temperature. After hydration, cells were incubated with 1% aqueous periodic acid and Schiff's reagent for 5 minutes each followed by washes in 0.5% sodium bisulphite water.²⁷

TABLE 1
Primer Sequences and RT-PCR Parameters

| | | | |
|---|---|---|---|
| | | PCR | |
| GENE | PRIMER SEQUENCE 5′-3′ | PARAMETERS^{A} | AMPLICON^{B} |

| | | | |
|---|---|---|---|
| HGF | F: AGGAGCCAGCCTGAATGATGA | 95, 56, 72 | 360 |
| | R: CCCTCTGATGTCCCAAGATTAGC | 1 M, 45 S, 1 M | |
| TGFα | F: ATGGTCCCCTCGGCTGGA | 95, 58, 72 | 297 |
| | R: GGCCTGCTTCTTCTGGCTGGCA | 45 S, 30 S, 1 M | |
| TGFβ1 | F: GCCCTGGACACCAACTATTGCT | 95, 58, 72 | 161 |
| | R: AGGCTCCAAATGTAGGGGCAGG | 45 A, 30 S, 1 M | |
| TGFβ2 | F: GATTTCCATCTACAAGACCACGAGGGACTTGC | 95, 58, 72 | 503 |
| | R: CAGCATCAGTTACATCGAAGGAGAGCCATTCG | 45 S, 30 S, 1 M | |
| HGFR | F: TGGTCCTTGGCGTCGTCCTC | 95, 54, 72 | 342 |
| | R: CTCATCATCAGCGTTATCTTC | 30 S, 45 S, 1 M | |
| EGFR | F: CTACCACCACTCTTTGAACTGGACCAAGG | 95, 58, 72 | 675 |
| | R: TCTATGCTCTCACCCCGTTCCAAGTATCG | 45 S, 30 S, 1 M | |
| TGFβ1R | F: CGTGCTGACATCTATGCAAT | 95, 54, 72 | 251 |
| | R: AGCTGCTCCATTGGCATAC | 30 S, 45 S, 1 M | |
| TGFβ2R | F: TGCACATCGTCCTGTGGAC | 95, 58, 72 | 784 |
| | R: GTCTCAAACTGCTCTGAAGTGTTC | 45 S, 30 S, 1 M | |
| FGFR | F: ATGTGGAGCTGGAAGTGCCTC | 95, 54, 72 | 165 |
| | R: GGTGTTATCTGTTTCTTTCTCC | 30 S, 45 S, 1 M | |
| IGF-1R | F: ACCCGGAGTACTTCAGCGCT | 95, 54, 72 | 229 |
| | R: CACAGAAGCTTCGTTGAGAA | 30 S, 45 S, 1 M | |
| HNF1α | F: GTGTCTACAACTGGTTTGCC | 95, 52, 72 | 251 |
| | R: TGTAGACACTGTCACTAAGG | 45 S, 30 S, 1 M | |
| HNF1β | F: GAAACAATGAGATCACTTCCTCC | 95, 56, 72 | 374 |
| | R: CTTTGTGCAATTGCCATGACTCC | 1 M, 45 S, 1 M | |
| HNF3β | F: CACCCTACGCCTTAACCAC | 95, 56, 72 | 235 |
| | R: GGTAGTAGGAGGTATCTGCGG | 1 M, 45 S, 1 M | |
| HNF4 | F: CTGCTCGGAGCCACAAAGAGATCCATG | 95, 58, 72 | 363 |
| | R: ATCATCTGCCACGTGATGCTCTGCA | 45 S,30 S, 1 M | |
| C/EBPα | F: CAAGAAGTCGGTGGACAAGAAC | 95, 58, 72 | 450 |
| | R: CCTCATCTTAGACGCACCAAGT | 45 S, 30 S, 1 M | |
| C/EBPβ | F: GCAACGCCGAGAGAGGA | 95, 58, 72 | 252 |
| | R: TGTCCTGCATTGTCGCC | 45 S, 30 S, 1 M | |
| GAPDH | F: CCATGGAGAAGGCTGGGG | 95, 58, 72 | 196 |
| | R: CAAAGTTGTCATGGATGACC | 45 S, 30 S, 1 M | |

C/EBP, CCAAT/enhancer binding protein; EGFR, epidermal growth factor receptor; F, forward primer; FGFR, fibroblast growth factor receptor; GAPDH, glyceraldehyde phosphate dehydrogenase; HGF, hepatocyte growth factor; HGFR, hepatocyte growth factor receptor; HNF, hepatocyte nuclear factor; IGF-1R, insulin-like growth factor-type I receptor; m, minutes; R, reverse primer; s, seconds; TGF, transforming growth factor; TGFR;transforming growth factor receptor.
^{a}Temperatures are tabulated in the first lane in ° C. and the corresponding times in the second lane. Reverse transcription was carried out at 42° C. for 20 minutes with a pre-PCR denaturation at 95° C. for. 10 minutes.

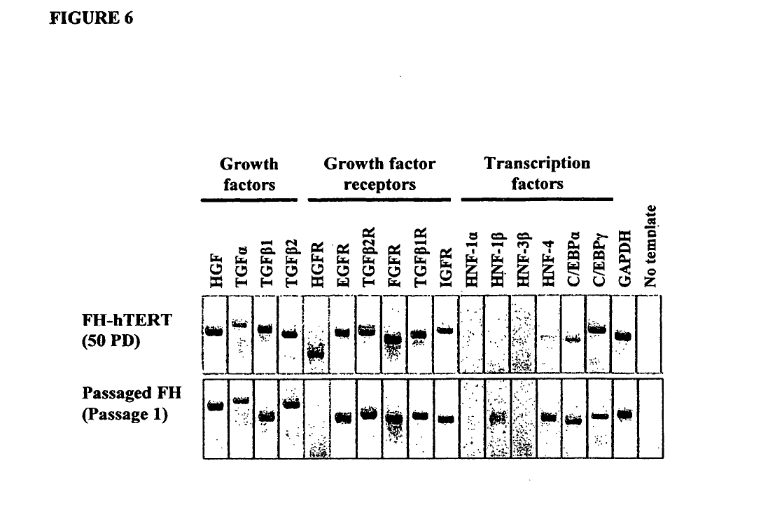

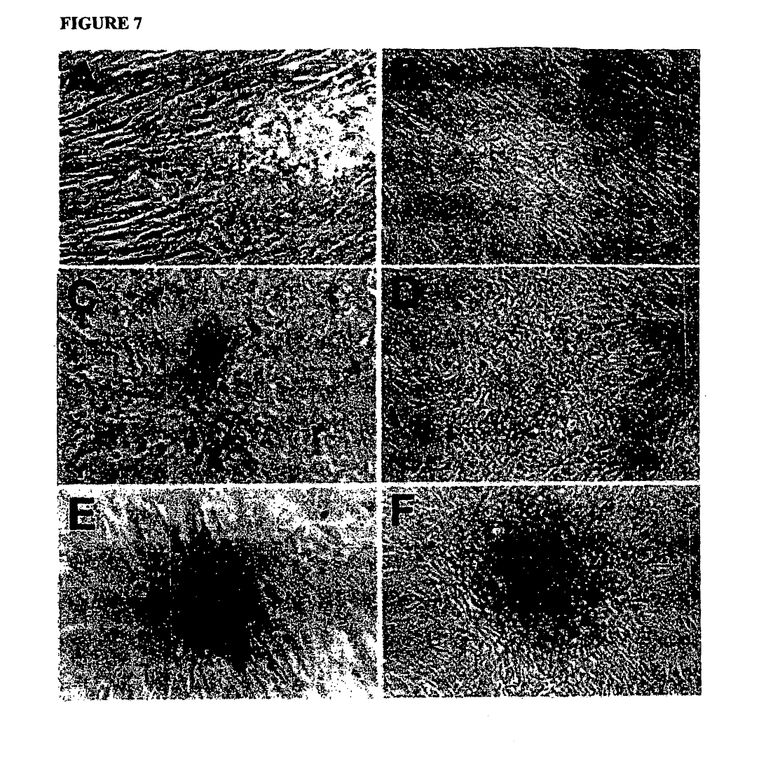

To address whether FH-hTERT maintained hepatocellular functions, transduced cells were examined in comparison with primary and passaged control FH. First, the expression profiles of various hepatic growth factors, growth factor receptors, and transcription factors were established. It is noteworthy that most genes examined were expressed in passaged FH (passage 1), as well as in FH-hTERT after 50 PD (FIG. 6). However, passaged FH showed a decrease in hepatocyte growth factor receptor (HGFR) expression (FIG. 6). Moreover, in these cells CCAAT/enhancer binding protein (C/EBP) α and hepatocyte nuclear factor (HNF) 4 expression were virtually undetectable in the fourth and subsequent cell passages (data not shown). In contrast, HGFR expression was upregulated in FH-hTERT at 50 PD, and these cells also continued to express important hepatocyte differentiation factors, especially C/EBPα and HNF-4 (FIG. 6). Members of the C/EBP gene family regulate both metabolism and cell growth and are predominantly expressed in tissues that conduct gluconeogenesis (Bucher and Farmer. In: Strain A J, Diehl A M, eds. Liver Growth and Repair. 1st ed. London: Chapman & Hall, 1998: 3-27). Therefore, we investigated glucose metabolism in FH-hTERT. Histochemistry revealed glycogen storage and glucose-6-phosphatase activity in confluent FH-hTERT at 50 PD, in a pattern similar to primary FH (FIG. 7). Next, we analyzed albumin production as a marker for hepatocellular protein synthesis by flow cytometry. Flow cytometry was employed as an assay to investigate FH-hTERT cultures on a single-cell level and to quantitate the percentage of cells with detectable albumin synthesis. At 60 PD, FH-hTERT exhibited a clear increase in green-fluorescence following albumin-specific fluorescein staining. The increase in green-fluorescence was similar to HepG2 cells (positive control) and indicated albumin synthesis in most of FH-hTERT. It should be noted that the culture conditions in our study were designed to support proliferation and have not been optimized for maintaining hepatocellular functions.

Hepatospecific gene expression, including albumin, α-fetoprotein, α1-antitrypsin, transferrin, and different cytochrome P450 subtypes, is compared in immortal versus freshly isolated human hepatocytes by real-time quantitative RT-PCR using Taqman chemistry (PE Applied Biosystems). Also, protein synthesis is measured by Western immunoblotting. Inducibility of cytochrome P450 2E1 subtype is evaluated by treating the cells with ethanol prior to expression analysis. Ureagenesis is investigated in a serum-free incubation system by measuring urea production and ammonium removal from the culture medium.

### Example 6

### Oncogenicity of Telomerase-Immortalized Cells

To determine serum dependence and contact inhibition, cells were cultured in serum-free medium for 5 days or in regular serum-supplemented growth medium until confluency for 15 days. The percentage of cells in S-phase was measured with the BrdU-incorporation assay specified above. Soft-agar colony assays were performed to evaluate anchorage-independent growth as described (Clark et al. Methods Enzymol 1995; 255: 395-412). Briefly, dilutions of 50, 500 and 5,000 cells in a 0.33% top agar were overlaid onto 0.5% base agar in 60-mm tissue culture dishes. Malignant HepG2 cells served as a positive control. After 15 days, colony formation was scored by counting under a microscope. Tumorigenicity was assessed by inoculating 10⁶ cells subcutaneously into the dorsal flanks, left and right of the midline, of male 6-8 week-old athymic nu/nu mice (Charles River Laboratories, Wilmington, Mass.) as approved by the institutional Animal Care and Use Administrative Advisory Committee of the University of California, Davis. Tumorigenic 293T cells were inoculated as a positive control and tumor formation was monitored twice weekly for 15 weeks.

To Analyze c-Myc Expression, protein was extracted, quantitated, and Western blot analysis of c-Myc was performed following published procedures (Liu et al. J. Biol Chem 2000; 51: 40155-40162), using a 1:100 dilution of mouse anti-c-Myc monoclonal antibody 9E10 (Santa Cruz Biotechnology, Santa Cruz, Calif.). To ensure equivalent amounts of protein loading, membranes were stripped with 0.2 M NaOH for 5 minutes and reprobed with goat anti-actin polyclonal antibody 1-19 (Santa Cruz Biotechnology) at a dilution of 1:200. Peroxidase-conjugated anti-mouse and anti-goat immunoglobulins (Santa Cruz Biotechnology) were diluted as directed by the manufacturer to immunodetect specific bands with ECL Western blotting detection reagents (Amersham Pharmacia Biotech, Piscataway, N.J.). Scanned Western blot images were quantitated densitometrically using ImageQuaNT (Molecular Dynamics) and c-Myc signals were normalized to actin and compared to passaged control FH as a calibrator.

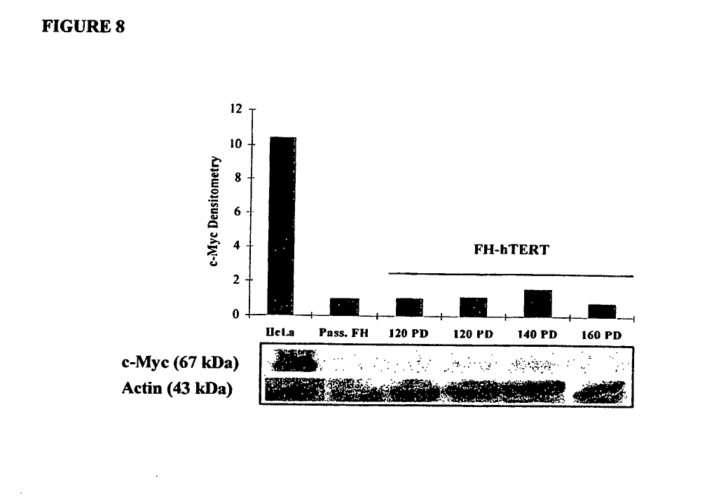

Cell culture assays were performed to investigate the oncogenicity of FH-hTERT. As expected for untransformed cells, DNA-synthesis rates of FH-hTERT significantly decreased from above 50% to less than 8% at confluency (contact inhibition) and during serum-starvation (Table 2). Anchorage-independent growth is also an excellent in vitro indicator of a malignant growth potential (Clark et al. Methods Enzymol 1995; 255: 395-412). Thus, we performed soft-agar colony assays with FH-hTERT and HepG2 cells. After 15 days, no colonies were observed in plates seeded with FH-hTERT (2 different cell pools with 3 plates per seeding density), whereas a seeding cell number-dependent colony formation was confirmed for HepG2 cells with 14±4 colonies for 50 seeded cells (n=4), 125±9 colonies for 500 seeded cells (n=4), and 830±71 colonies for 5,000 seeded cells (n=4). Next we inoculated FH-hTERT into immunodeficient athymic nude mice to investigate in vivo tumor formation. All mice inoculated with FH-hTERT (2 different cell pools with 3 mice per cell pool) showed no tumor formation within the observation period of 15 weeks. In the positive control group, 3 out of 4 mice had detectable tumors after a latency period of 5.1±1.0 weeks reaching tumor diameters of more than 10 mm 7.7-11.6 weeks after 293T cell inoculation. Furthermore, despite more than 160 PD, we observed no upregulation of c-Myc by Western blot analysis (FIG. 8). Densitometry of scanned c-Myc and actin bands revealed no changes for FH-hTERT after 160 PD. An increase in c-Myc was a feature of telomerase-immortalized human mammary epithelial cells cultured for less time by other investigators raising concerns of potential telomerase immortalization-related genetic changes.³¹

TABLE 2
Serum Dependence and Contact Inhibition

| | Percentage of cells in S-phase | | |
|---|---|---|---|
| Culture | FH-Control | FH-hTERT 1 | FH-hTERT 2 |
| condition | (24 PD) | (100 PD) | (120 PD) |
| 10% FBS, | 36.79 ± 0.53 | 52.93 ± 1.76 | 50.71 ± 2.23 |
| 1 day^{a} | (n = 3) | (n = 3) | (n = 4) |
| 10% FBS, | ND | 7.83 ± 0.85 | 6.15 ± 1.13 |
| confluent^{b} | | (n = 4)^{d} | (n = 4)^{d} |
| 0% FBS, | ND | 6.09 ± 0.74 | 5.43 ± 0.54 |
| 5 days^{c} | | (n = 4)^{d} | (n = 4)^{d} |

NOTE.
Values are means ± SEM. Data for 2 different FH-hTERT pools.
FBS, fetal bovine serum; ND, not determined.
^{a}overnight semi-confluent culture (1 day) in medium containing 10% FBS.
^{b}Prolonged confluent culture (15 days) in medium containing 10% FBS.
^{c}Short-term culture (5 days) in medium without FBS.
^{d}Significant difference from 10% FBS, 1 day (P < 0.001).

Growth requirements, karyotypic stability, and cell-cycle checkpoints are analyzed to detect any possible oncogenic potential of the immortalized hepatocytes The presence of chromosomal abnormalities is a fundamental feature of tumor progression. Karyotype analysis is conducted by G-banding in a cytogenetic laboratory. Western analysis for the hypo- and hyperphosphorylated form of retinoblastoma protein is performed after cell growth in low-serum medium (Jiang et al. (1999) Nat Genet 21: 111-114.). Finally, DNA damage is induced by UV-B irradiation (100 J/m²) and p53 is quantitated by Western immunoblotting.

### Example 7

### Immortalization of Human Adult Hepatocytes by the Ectopic Expression of Telomerase Reverse Transcriptase (TERT) Employing a Simian Virus 40-Based Vector System

One of our SV40-derived cloning targets, pT7A5Δ4.2, was used to design a TERT-delivery system. pT7A5Δ4.2 uses pT7 as carrier plasmid. The early and late SV40 genes are excised from the viral genome, including large T antigen, to make enough room for transgenes (up to 4.7 kilo bases) and to render the vector replication-deficient (Strayer 1996). A CMV intermediate early promoter was added to facilitate high levels of transgene expression. The polylinker of pT7A5Δ4.2 was modified to provide more convenient unique restriction sites for greater flexibility in cloning. TERT (3.5 kilo bases) was cut from the retroviral expression plasmid pBabe-puro-hTERT (provided by Dr. R. A. Weinberg, Whitehead Institute for Biomedical Research, Cambridge, Mass.) and cloned into pT7A5Δ4.2. To generate SV40-TERT recombinant virus, SV40-TERT sequences are excised from pT7A5Δ4.2, purified, recircularized, and transfected into COS-7 cells. These cells contain an integrated copy of the wild-type SV40 genome defective at the origin of replication. Thus, COS-7 cells function as packaging cells for our SV40-derived vector, providing essential viral proteins in trans. High viral titers can be harvested for the subsequent transduction of human adult hepatocytes (Strayer J. Biol Chem 271: 24741-24746, 1996).

### Example 8

### Evaluation of Proliferation and Differentiated States of Transduced and Untransduced Cells on the Components of BAL

Various foams and polymers are then tested that have been designed for incorporation of immortalized hepatocytes into a bioartificial liver device. An effective approach includes the initial testing of these substrates on a small scale in dishes. Initially, these assays are performed using rat hepatocytes. Briefly, to evaluate the foam materials for biocompatibility and the effect on differentiated function of primary hepatocytes, rats are used to isolate hepatocytes and other cell types in the liver. One preparation of cells is enough to evaluate several foam samples. Using isolated rat liver cells allows the determination of the gross trends without using human cells. An established two-step collagenase digestion method is used for isolation of rat hepatocytes. A rat is anesthetized with pentobarbital (60 mg/kg, i.p.) before opening the abdomen and inserting a catheter into the portal vein for liver perfusion. The liver is perfused with Ca²⁺-free perfusion butter and then with a solution containing collagenase (0.5 mg/ml) to digest the liver in situ. The digested liver is dissected, filtered and washed with a low speed centrifugation at 4° C.

Materials and surface coatings are first evaluated with rat hepatocytes, and then refined with human hepatocytes. The assays involve cell attachment during the first hour and after overnight culture, preservation of cell viability with thiazolyl blue (MTT) dye utilization assays, cell doubling times, time to confluency, as well as growth factor responsiveness with incorporation of ³H-thymidine into TCA-precipitable DNA. RT-PCR studies are conducted by extracting RNA from cultured cells to determine which growth factor receptors are expressed in cells cultured on various target matrices. For this purpose, panels of available specific primer sequences, are used for testing expression of EGF, HGF, FGF, TGF-alpha and other growth factor receptors. Studies are conducted to test incorporation of specific growth factors, such as TGF-alpha, EGF, HGF, various hormones and other additives to demonstrate whether the proliferation rates can be optimized for rapid seeding of the devices. While information is available concerning use of defined media, such as Block's medium for rat hepatocytes (Block et al. (1996) J. Cell Biol 132: 1133-49), it is unknown whether fetal human hepatoblasts or adult hepatocytes will respond in an identical fashion. Cells may well require different culture conditions to optimize various aspects of their growth on the components of the bioartificial organ. For example, different media may enhance cell attachment, but not be optimal for survival, proliferation, or hepatospecific gene function. Thus, empirical studies are required to optimize this process before other components of the bioreactor are finalized. Similarly, a variety of extracellular matrix components must be tried in an empiric fashion to optimize cell attachment, proliferation, and function. Finally, the value of coculturing with endothelial cells is also investigated.

### Example 9

### Effectiveness of Immortalized Hepatocytes in the BAL

The most effective immortalized hepatocytes are then employed in the BAL. To undertake studies with the actual bioartificial liver, it is necessary to seed the devices and analyze rates of cell attachment, survival and proliferation at predetermined intervals, such as 1, 3, 7, 14, and 28 days. This requires in situ labeling of cells followed by disassembly of the device and morphologic analysis. To demonstrate that the bioartificial livers permit appropriate expression of liver cell genes, studies are conducted with western and northern blots, metabolic labeling, gel shifts and other methods. The expectation is that under suitable conditions, when proliferative activity of the cells is no longer required, it is then possible to induce liver-specific function in the immobilized cells. This necessitates testing of various surface coatings on the porous scaffolds, such as Matrigel, collagens, laminin, fibronectin, etc. Conceivably, confluency can promote differentiation by itself. The assays will include quantitation of liver gene expression by histochemical methods in cells fixed in situ, as well as with specific antibody or molecular probes with total cellular RNA or protein extracts. Upon seeding of the bioartificial liver device, timed collections are made to demonstrate secretion of proteins, such as albumin and alpha-1 macroglobulin. Also, activation of P450-inducible gene expression patterns is demonstrated by established assays, where phenobarbital and clofibrate-induction of P450 activity is tested.

### Example 10

### In Vivo Maturation of Telomerase-Immortalized Human Fetal Hepatocytes (FH-hTERT)

2×10⁶ immortalized cells (after 150 population doublings in culture) were transplanted via portal vein injection into the livers of partially hepatectomized NOD-SCID mice. Four weeks later, transplanted mice were sacrificed and total RNA was extracted. Real-time quantitative RT-PCR technology was used to amplify human-specific albumin, α1-antitrypsin, transferrin, and glyceraldehyde phosphate dehydrogenase as endogenous control. Hepatectomized mouse liver tissue without human cells served as a negative control to verify human-specific amplification. Applying a comparative quantification method (ΔΔCt), expression levels were normalized to the endogenous control and calibrated to human hepatocyte samples.

We observed upregulation for all investigated human hepatocyte-specific genes, reaching almost human hepatocyte expression levels; in particular, 13.6±2.69% (mean expression level from 3 mice with 3 RNA extractions per mice±SEM relative to human hepatocytes) for albumin, 57.6±10.28% for α1-antitrypsin, and 30.4±12.34% for transferrin.

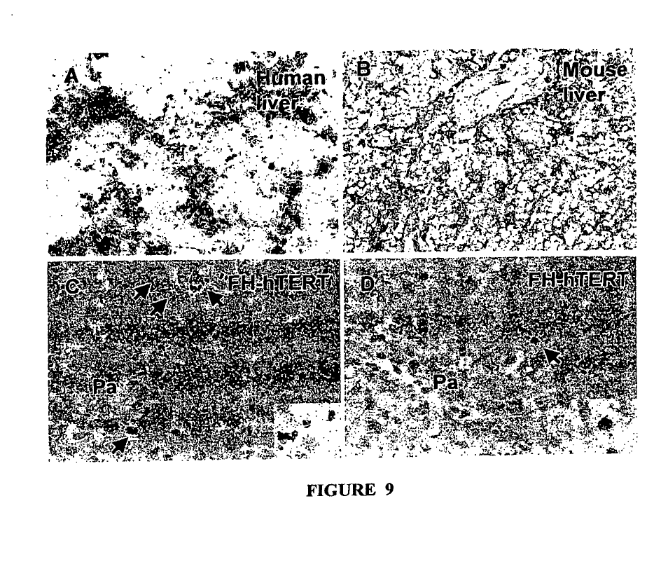

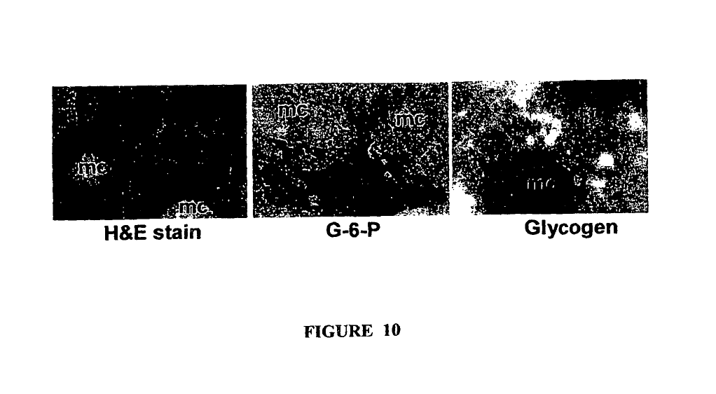

Engraftment of our cells in the livers of NOD-SCID mice was further confirmed by amplification of human-specific Alu-sx sequences (data not shown) and by in situ hybridization using a human-specific centromer probe (see FIG. 9). Further confirmation of the ability of our cells to produce a liver-specific phenotype is provided from experiments when the FH-hTERT cells were transplanted intraperitoneally into NOD-SCID mice on microcarrier beads and recovered for histological analyses (see FIG. 10).

As suggested by our previous data showing the uninterrupted expression of important hepatocyte differentiation factors, in particular hepatocyte nuclear factor 4 and CCAAT/enhancer binding protein α, and the data summarized above, telomerase-immortalized human fetal hepatocytes maintain their ability to mature in vivo despite prolonged time in culture. The ability to engraft in mouse liver tissue and the upregulation of hepatocellular proteins in vivo, indicate that telomerase-immortalization does not disturb endogenous differentiation programs.

There is no evidence of tumorigenicity in FH-hTERT. Inoculation of 6 nu/nu mice with FH-hTERT led to no tumor formation after four months of observation, whereas 75% of mice inoculated with 293T cells developed tumors after 5.05±0.96 weeks. Moreover, preliminary findings demonstrate that FH-hTERT cells are amenable to blockade with specific drugs, such as nocadozole (G2/M arrest), hydroxyure (late S arrest) and aphidocolin (G1/S arrest), as well as radiation (G0/G1 and G2/M arrest). The results are compatible with intact p53 function and an untransformed phenotype.

### Example 11

### Immortalization of Nestin-Positive Islet-Derived Progenitor (NIP) Cells by the Ectopic Expression of Human Telomerase Reverse Transcriptase (hTERT)

Transduction: NIP cells are transduced with an amphotrophic retroviral vector carrying hTERT and puromycin acetyltransferase as a selectable marker. The vector is produced by cotransfection (lipofection) of 293T cells with the expression plasmid pBabe-puro-hTERT and the packaging plasmid pCl-Ampho. NIP cells are transduced with full-strength viral supernatant from 48 and 72 hour harvests. Using a 293T cell-based bioassay, the viral titer is determined to be between 10³ and 10⁴ puromycin-resistant colony-forming units per milliliter of supernatant. NIP cells are transduced in 25 cm³ culture vessels on two consecutive days. Transduction is carried out for 3 hours each day and polybrene (8 μg/ml) is used to increase transduction efficiency.

Selection: The optimal puromycin concentration to select NIP cells is determined in a puromycin cytotoxicity experiment using phase contrast microscopy to evaluate cellular viability (typically between as 0.75 to 1 μM). After a recovery period of 3 days post transduction, transduced NIP cells are selected for more than 3 weeks until completion. Selected cells are designated NIP-hTERT and reseeded to expand the cell number for phenotype and tumorigenicity analyses.

hTERT Expression: Expression of hTERT is measured using the LightCycler TeloTAGGGhTERT Quantification Kit (Roche Molecular Biochemicals), a real time quantitative RT-PCR protocol. Human porphobilinogen deaminase (PBGD) is used as a housekeeping control to normalize hTERT expression. Untransduced NIP cells have undetectable hTERT expression, while NIP-hTERT have 11.9 copies of hTERT per copy of PBGD. This level of hTERT expression is similar to that in telomerase-positive cancer cell lines.

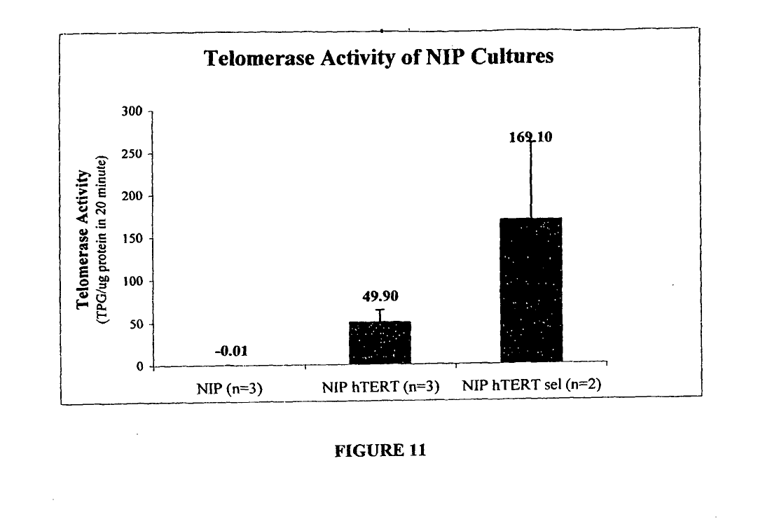

Telomerase Activity: Telomerase activity is measured using the TeloTAGGG Telomerase PCR ELISA (Roche Molecular Biochemicals), a commercially available telomeric repeat amplification protocol kit. Telomerase activity is measured twice, once during and once after complete selection. Untransduced NIP cells have undetectable telomerase activity. All NIP-hTERT cultures, during and after selection, have high telomerase activity ranging from 23.36 to 169.1 total product generated per ug of protein in 20 minutes reaction time (see FIG. 11).

All publications and patent applications mentioned in this specification are indicative of the level of skill of those skilled in the art to which this invention pertains. All publications and patent applications are herein incorporated by reference to the same extent as if each individual publication or patent application was specifically and individually indicated to be incorporated by reference.

The invention now having been fully described, it will be apparent to one of ordinary skill in the art that many changes and modifications can be made thereto without departing from the spirit or scope of the appended claims.

1. An immortalized cell, wherein said cell comprises:
a functional telomerase catalytic subunit, wherein said cell maintains at least one function specific to the cell type from which it was derived.

2. The immortalized cell according to claim 1, wherein said functional telomerase catalytic subunit is encoded by a human TERT gene.

3. The immortalized cell according to claim 1, wherein said cell is a transplantable cell.

4. The immortalized cell according to claim 3, wherein said cell is a hepatocyte or a pancreatic cell.

5. The immortalized cell according to claim 4, wherein said cell is a fetal cell.

6. The immortalized fetal cell according to claim 5, wherein % aid cell is transduced with a retrovirus.

7. The immortalized cell according to claim 4, wherein said cell is a hepatocyte and said function is (a) expression of one or more protein selected from the group consisting of albumin, α-fetoprotein, α1-antitrypsin, transferrin, HGF, TGFα, TGFβ1, TGFβ2, HGFR, EGFR, TGFβ1R, TGFβ2R, FGFR, IGF-1R, HNF1α, HNF1β, HNF3β, HNF4, C/EBPα, C/EBPβ, GAPDH; (b) inducibility of cytochrome P450 2E1 and (c) ureagenesis.

8. The immortalized cell according to claim 4, wherein said cell is a pancreatic cell and said function is expression of at least one protein selected from the group consisting of GLUT2, insulin, glucagon, and IDX-1.

9. A non-senescent, non-transformed immortalized human cell, wherein said cell has been cultured for at least about 35 population doublings.

10. An immortalized human cell according to claim 9, wherein said cell maintains a telomere length of at least about 6 kb.

11. A bioartificial organ comprising:
a pluarility of said immortalized cell according to claim 1.

12. A method of obtaining an immortalized human cell, said method comprising the steps of:
transducing a human cell with a nucleic acid encoding a functional telomerase catalytic subunit, whereby a transduced human cell is obtained; and
growing said transduced human cell so that said nucleic acid is expressed, whereby an immortalized human cell is obtained that maintains at least one function specific to said human cell from which it was derived.

13. The method according to claim 12, wherein said human cell is a hepatocyte or a pancreatic cell.

14. The method according to claim 12 or claim 13, wherein said human cell is a fetal cell.

15. The method according to claim 12, wherein said fetal human cell is step of transducing is with a retrovirus.

16. A bioartificial organ comprising:
a capsule and a plurality of said immortalized human cell according to claim 1.

17. A method of ameliorating at least one symptom of a metabolic disease in an individual in need thereof, said method comprising the step of:
perfusing blood of said individual through a bioartificial organ according to claim 16, whereby at least one symptom is ameliorated.

18. A method of ameliorating at least one symptom of hepatic disease in an individual in need thereof, said method comprising the step of:
transplanting to said individual a plurality of immortalized human hepatocytes that express a functional telomerase catalytic subunit and maintain at least one specific hepatic function, whereby at least one symptom of said hepatic disease is ameliorated.

19. An immortalized pancreatic cell comprising:
a progenitor cell that expresses a functional telomerase catalytic subunit and maintains at least one specific pancreatic cell function.

20. The immortalized pancreatic cell according to claim 19, wherein said progenitor cell is derived from a nestin-positive islet cell.

21. The immortalized pancreatic cell according to claim 19, wherein said immortalized cell produces biologically active insulin.

22. The immortalized pancreatic cell according to claim 21, wherein said biologically active insulin is produced in response to glucose stimulation.

23. An immortalized pancreatic cell line comprising:
a plurality of said immortalized pancreatic cell according to claim 22.

24. An immortalized pancreatic cell line comprising a plurality of said immortalized pancreatic cell according to claim 21.

25. A method of treating at least one symptom of diabetes in a patient in need thereof said method comprising:
transplanting into said patient a plurality of said immortalized pancreatic cell according to claim 21.


SEQUENCE LISTING (35 sequences)

SEQ ID NO: 1 - DNA, 21 bp, Artificial Sequence
  Description of Artificial Sequencehepatocyte growth factor (HGF) RT-PCR forward primer
aggagccagcctgaatgatga

SEQ ID NO: 2 - DNA, 23 bp, Artificial Sequence
  Description of Artificial Sequencehepatocyte growth factor (HGF) RT-PCR reverse primer
ccctctgatgtcccaagattagc

SEQ ID NO: 3 - DNA, 18 bp, Artificial Sequence
  Description of Artificial Sequencetransforming growth factor alpha (TGFalpha) RT-PCR forward primer
atggtcccctcggctgga

SEQ ID NO: 4 - DNA, 22 bp, Artificial Sequence
  Description of Artificial Sequencetransforming growth factor alpha (TGFalpha) RT-PCR reverse primer
ggcctgcttcttctggctggca

SEQ ID NO: 5 - DNA, 22 bp, Artificial Sequence
  Description of Artificial Sequencetransforming growth factor beta 1 (TGFbeta1) RT-PCR forward primer
gccctggacaccaactattgct

SEQ ID NO: 6 - DNA, 22 bp, Artificial Sequence
  Description of Artificial Sequencetransforming growth factor beta 1 (TGFbeta1) RT-PCR reverse primer
aggctccaaatgtaggggcagg

SEQ ID NO: 7 - DNA, 32 bp, Artificial Sequence
  Description of Artificial Sequencetransforming growth factor beta 2 (TGFbeta2) RT-PCR forward primer
gatttccatctacaagaccacgagggacttgc

SEQ ID NO: 8 - DNA, 32 bp, Artificial Sequence
  Description of Artificial Sequencetransforming growth factor beta 2 (TGFbeta2) RT-PCR reverse primer
cagcatcagttacatcgaaggagagccattcg

SEQ ID NO: 9 - DNA, 20 bp, Artificial Sequence
  Description of Artificial Sequencehepatocyte growth factor receptor (HGFR) RT-PCR forward primer
tggtccttggcgtcgtcctc

SEQ ID NO: 10 - DNA, 21 bp, Artificial Sequence
  Description of Artificial Sequencehepatocyte growth factor receptor (HGFR) RT-PCR reverse primer
ctcatcatcagcgttatcttc

SEQ ID NO: 11 - DNA, 29 bp, Artificial Sequence
  Description of Artificial Sequenceepidermal growth factor receptor (EGFR) RT-PCR forward primer
ctaccaccactctttgaactggaccaagg

SEQ ID NO: 12 - DNA, 29 bp, Artificial Sequence
  Description of Artificial Sequenceepidermal growth factor receptor (EGFR) RT-PCR reverse primer
tctatgctctcaccccgttccaagtatcg

SEQ ID NO: 13 - DNA, 20 bp, Artificial Sequence
  Description of Artificial Sequencetransforming growth factor beta 1 receptor (TGFbeta1R) RT-PCR forward primer
cgtgctgacatctatgcaat

SEQ ID NO: 14 - DNA, 19 bp, Artificial Sequence
  Description of Artificial Sequencetransforming growth factor beta 1 receptor (TGFbeta1R) RT-PCR reverse primer
agctgctccattggcatac

SEQ ID NO: 15 - DNA, 19 bp, Artificial Sequence
  Description of Artificial Sequencetransforming growth factor beta 2 receptor (TGFbeta2R) RT-PCR forward primer
tgcacatcgtcctgtggac

SEQ ID NO: 16 - DNA, 24 bp, Artificial Sequence
  Description of Artificial Sequencetransforming growth factor beta 2 receptor (TGFbeta2R) RT-PCR reverse primer
gtctcaaactgctctgaagtgttc

SEQ ID NO: 17 - DNA, 21 bp, Artificial Sequence
  Description of Artificial Sequencefibroblast growth factor receptor (FGFR) RT-PCR forward primer
atgtggagctggaagtgcctc

SEQ ID NO: 18 - DNA, 22 bp, Artificial Sequence
  Description of Artificial Sequencefibroblast growth factor receptor (FGFR) RT-PCR reverse primer
ggtgttatctgtttctttctcc

SEQ ID NO: 19 - DNA, 20 bp, Artificial Sequence
  Description of Artificial Sequenceinsulin-like growth factor-type 1 receptor (IGF-1R) RT-PCR forward primer
acccggagtacttcagcgct

SEQ ID NO: 20 - DNA, 20 bp, Artificial Sequence
  Description of Artificial Sequenceinsulin-like growth factor-type 1 receptor (IGF-1R) RT-PCR reverse primer
cacagaagcttcgttgagaa

SEQ ID NO: 21 - DNA, 20 bp, Artificial Sequence
  Description of Artificial Sequencehepatocyte nuclear factor 1alpha (HNF1alpha) RT-PCR forward primer
gtgtctacaactggtttgcc

SEQ ID NO: 22 - DNA, 20 bp, Artificial Sequence
  Description of Artificial Sequencehepatocyte nuclear factor 1alpha (HNF1alpha) RT-PCR reverse primer
tgtagacactgtcactaagg

SEQ ID NO: 23 - DNA, 23 bp, Artificial Sequence
  Description of Artificial Sequencehepatocyte nuclear factor 1beta (HNF1beta) RT-PCR forward primer
gaaacaatgagatcacttcctcc

SEQ ID NO: 24 - DNA, 23 bp, Artificial Sequence
  Description of Artificial Sequencehepatocyte nuclear factor 1beta (HNF1beta) RT-PCR reverse primer
ctttgtgcaattgccatgactcc

SEQ ID NO: 25 - DNA, 19 bp, Artificial Sequence
  Description of Artificial Sequencehepatocyte nuclear factor 3beta (HNF3beta) RT-PCR forward primer
caccctacgccttaaccac

SEQ ID NO: 26 - DNA, 21 bp, Artificial Sequence
  Description of Artificial Sequencehepatocyte nuclear factor 3beta (HNF3beta) RT-PCR reverse primer
ggtagtaggaggtatctgcgg

SEQ ID NO: 27 - DNA, 27 bp, Artificial Sequence
  Description of Artificial Sequencehepatocyte nuclear factor 4 (HNF4) RT-PCR forward primer
ctgctcggagccacaaagagatccatg

SEQ ID NO: 28 - DNA, 25 bp, Artificial Sequence
  Description of Artificial Sequencehepatocyte nuclear factor 4 (HNF4) RT-PCR reverse primer
atcatctgccacgtgatgctctgca

SEQ ID NO: 29 - DNA, 22 bp, Artificial Sequence
  Description of Artificial SequenceCCAAT/enhancer binding protein alpha (C/EBPalpha) RT-PCR forward primer
caagaagtcggtggacaagaac

SEQ ID NO: 30 - DNA, 22 bp, Artificial Sequence
  Description of Artificial SequenceCCAAT/enhancer binding protein alpha (C/EBPalpha) RT-PCR reverse primer
cctcatcttagacgcaccaagt

SEQ ID NO: 31 - DNA, 17 bp, Artificial Sequence
  Description of Artificial SequenceCCAAT/enhancer binding protein beta (C/EBPbeta) RT-PCR forward primer
gcaacgccgagagagga

SEQ ID NO: 32 - DNA, 17 bp, Artificial Sequence
  Description of Artificial SequenceCCAAT/enhancer binding protein beta (C/EBPbeta) RT-PCR reverse primer
tgtcctgcattgtcgcc

SEQ ID NO: 33 - DNA, 18 bp, Artificial Sequence
  Description of Artificial Sequenceglyceraldehyde phosphate dehydrogenase (GAPDH) RT-PCR forward primer
ccatggagaaggctgggg

SEQ ID NO: 34 - DNA, 20 bp, Artificial Sequence
  Description of Artificial Sequenceglyceraldehyde phosphate dehydrogenase (GAPDH) RT-PCR reverse primer
caaagttgtcatggatgacc

SEQ ID NO: 35 - DNA, 18 bp, Artificial Sequence
  Description of Artificial Sequence32-P labeled telomeric probe
ttagggttagggttaggg